(19) United States
(12) Patent Application Publication
(10) Pub. No.: US 2025/0177650 A1
(43) Pub. Date: Jun. 5, 2025

(54) VISION BASED AUTOMATED SYRINGE VOLUME MEASUREMENT

(71) Applicant: Omnicell, Inc., Mountain View, CA (US)

(72) Inventors: Oleg Lapets, Wexford, PA (US); Christopher Smith, Hermitage, PA (US)

(73) Assignee: Omnicell, Inc., Mountain View, CA (US)

(21) Appl. No.: 18/961,336

(22) Filed: Nov. 26, 2024

Related U.S. Application Data
(60) Provisional application No. 63/604,733, filed on Nov. 30, 2023.

(51) Int. Cl. A61M 5/20; A61B 34/30; A61B 90/00

(52) U.S. Cl. CPC A61M 5/20; A61B 34/30; A61B 90/06; A61B 90/37; A61B 2034/305; A61B 2090/063; A61M 2005/2006

(57) ABSTRACT

The present relates to vision based automated syringe volume measurement. This can include a method and system for syringe volume measurement. The method can include filling a volume of liquid into a syringe, which volume of liquid is unknown, positioning the syringe for imaging with a high-resolution camera, capturing an image of the syringe with the high-resolution camera, and evaluating the image of the syringe to determine the volume of liquid in the syringe.

### CROSS-REFERENCES TO RELATED APPLICATIONS

This Non-Provisional Application claims the benefit of priority to U.S. Provisional Application No. 63/604,733, entitled “Vision Based Automated Syringe Volume Measurement”, filed Nov. 30, 2023, the entirety of which is hereby incorporated by reference herein.

The present application is related to: U.S. patent application Ser. No. 17/005,786, filed Aug. 28, 2020, and entitled “Medication Dosing Systems And Methods”; U.S. patent application Ser. No. 17/005,650, filed Aug. 28, 2020, and entitled “Cartridge Loading System for Syringe Caps; U.S. patent application Ser. No. 17/005,637, filed Aug. 28, 2020, and entitled “Bag Transfer Mechanism”; and U.S. patent application Ser. No. 17/006,027, filed Aug. 28, 2020, and entitled “Systems And Methods For Parallel Preparation Processing” the disclosures of each, which are hereby incorporated by reference in their entireties for all purposes.

### BACKGROUND

Pharmaceutical compounding is the preparation of medications by the processing or combination of ingredients. Many medications, especially medications administered orally in pill form, are now manufactured in a variety of forms and dosages so that little preparation is needed at a pharmacy, other than placing the proper number of pills in a bottle to fill a doctor's prescription for a particular patient. However, medications for intravenous delivery are routinely compounded in hospital pharmacies or specialty clinics. Compounded medications may be patient-specific, or frequently used medications may be prepared and stocked for later use.

Part of the preparation of medications can include the loading of a syringe. This loading can include aspirating a volume medication in form of a liquid into a syringe. This volume of medication can then be administered to a patient via an injection, or can be injected into an intravenous fluid bag for subsequent intravenous delivery to a patient.

It is of utmost importance that the correct medications be prepared in the correct proportions, without the introduction of contaminants. With respect to a syringe, this includes ensuring that the proper amount of medication is loaded into the syringe. Determining that the proper amount of medication is loaded into the syringe can be time consuming and can introduce risk of error.

### BRIEF SUMMARY

Aspects disclosed herein relate to vision based automated syringe volume measurement. This can include a method of syringe volume measurement. The method can include filling a volume of liquid into a syringe, which volume of liquid is unknown, positioning the syringe for imaging with a high-resolution camera, capturing an image of the syringe with the high-resolution camera, and evaluating the image of the syringe to determine the volume of liquid in the syringe.

In some embodiments, the method includes filling air into the syringe to create an air gap between a meniscus of the liquid in the syringe and a top of the syringe. In some embodiments, positioning the syringe for imaging includes positioning the syringe in a vertical orientation with the top above the plunger. In some embodiments, positioning the syringe for imaging includes holding the syringe with a robotic arm.

In some embodiments, evaluating the image includes determining a seal position, and determining the volume of liquid in the syringe based on the seal position. In some embodiments, evaluating the image further includes determining a location of a liquid meniscus in the image of the syringe, and determining a distance between the location of the liquid meniscus and the seal position. In some embodiments, the volume of liquid in the syringe is determined based on the distance between the location of the liquid meniscus and the seal position. In some embodiments, determining the distance between the location of the liquid meniscus and the seal position includes counting a number of pixels in a shortest straight line between the seal position and the location of the liquid meniscus.

In some embodiments, determining the location of the liquid meniscus includes defining a region of interest (“ROI”) in the image of the syringe, and creating a sub-image based on the image of the syringe and on the ROI. In some embodiments, determining the location of liquid meniscus includes performing a variance-based convolution on the sub-image, calculating horizontal projection of pixels with intensities above a threshold value, and identifying a first peak in the horizontal projection of pixels above the seal position. In some embodiments, the method includes identifying the location of the first peak in the horizontal projection of pixels as the location of the liquid meniscus.

In some embodiments, evaluating the image further includes converting the image of the syringe to a black-and-white image, and creating a binary image from the black-and-white image. In some embodiments, the binary image is created from the black-and-white image via thresholding. In some embodiments, the seal position is determined in the binary image.

In some embodiments, the method includes identifying the air gap based on the image of the syringe, and adjusting the plunger position in the syringe to eliminate the air gap. In some embodiments, identifying the air gap includes evaluating the image of the syringe to identify a top of the syringe, and determining a distance between the liquid meniscus and the top of the syringe.

The system for syringe volume measurement can include a syringe loading station, a syringe holder, a high-resolution camera configured to image the syringe loading station, and a processor. The processor can fill with the syringe loading station a volume of liquid into a syringe, which volume of liquid is unknown, position with the syringe holder the syringe for imaging with the camera, capture with the camera an image of the syringe with the high-resolution camera, and evaluate the image of the syringe to determine the volume of liquid in the syringe.

In some embodiments, the processor can further fill air into the syringe with the syringe loading station to create an air gap between a meniscus of the liquid in the syringe and a top of the syringe. In some embodiments, positioning the syringe for imaging includes positioning the syringe in a vertical orientation with the top above the plunger. In some embodiments, positioning the syringe for imaging includes holding the syringe with a robotic arm.

In some embodiments, evaluating the image includes determining a seal position, and determining the volume of liquid in the syringe based on the seal position. In some embodiments, evaluating the image further includes determining a location of a liquid meniscus in the image of the syringe, and determining a distance between the location of the liquid meniscus and the seal position. In some embodiments, the volume of liquid in the syringe is determined based on the distance between the location of the liquid meniscus and the seal position. In some embodiments, determining the distance between the location of the liquid meniscus and the seal position includes counting a number of pixels in a shortest straight line between the seal position and the location of the liquid meniscus.

In some embodiments, determining the location of the liquid meniscus includes defining a region of interest (“ROI”) in the image of the syringe, and creating a sub-image based on the image of the syringe and on the ROI. In some embodiments, determining the location of liquid meniscus includes performing a variance-based convolution on the sub-image, calculating horizontal projection of pixels with intensities above a threshold value, and identifying a first peak in the horizontal projection of pixels above the seal position.

In some embodiments, the processor can further identify the location of the first peak in the horizontal projection of pixels as the location of the liquid meniscus. In some embodiments, evaluating the image further includes converting the image of the syringe to a black-and-white image, and creating a binary image from the black-and-white image. In some embodiments, the binary image is created from the black-and-white image via thresholding. In some embodiments, the seal position is determined in the binary image.

In some embodiments, the processor can further identify the air gap based on the image of the syringe, and adjust the plunger position in the syringe to eliminate the air gap. In some embodiments, identifying the air gap includes evaluating the image of the syringe to identify a top of the syringe, and determining a distance between the liquid meniscus and the top of the syringe.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic depiction of one embodiment of a system for vision-based automated syringe volume measurement.

FIG. 2 is a schematic depiction of a camera positioned to image a syringe in a syringe loading station.

FIG. 3 is a depiction of a syringe held by a robotic-arm and being imaged by a camera.

FIG. 4 is a flowchart illustrating one embodiment of a process for vision-based automated syringe volume measurement.

FIG. 5 is a flowchart illustrating one embodiment of a process for evaluating an image of a syringe to determine a syringe volume.

FIG. 6 is a flowchart illustrating one embodiment of a process for determining a location of a liquid meniscus in the syringe.

FIG. 7 includes images showing an image of a syringe in raw form, as a black-and-white image, and as a binary image.

FIG. 8 includes images depicting one embodiment of a process for identifying a seal position.

FIG. 9 includes images depicting one embodiment of a process of creating a sub-image.

FIG. 10 includes images depicting one embodiment of a process for determining a liquid meniscus location.

FIG. 11 includes an image depicting the distance between the seal position and the liquid meniscus location of a syringe.

FIG. 12 is a schematic illustration of on embodiment of a computer system.

### DETAILED DESCRIPTION

Automated syringe-based compounding can be performed by a compounding station, and relies on a precise measurement of the amount of each drug aspirated into the syringe. At present, the required measurement precision is achieved by using a gravimetric method. In such an embodiment, a syringe loaded with a medication is placed on a high precision scale. In some embodiments, this weight alone is used to calculate the amount of medication in the syringe, whereas in other embodiments, a combination of this weight and a known medication density are used to calculate the amount of medication in the syringe.

This gravimetric approach can produce good results under ideal circumstances, such as when this approach is performed within an undisturbed environment. However, even minor disturbances can adversely affect the efficacy of the gravimetric approach. For example, environment disturbance such as air flow may interfere with the electronic scale and, thus, cause a deviation from a true measurement thereby adversely affecting the accuracy of the measurement. Other minor disturbances that can affect the accuracy of the measurement include vibrations, electric and/or magnetic fields, and/or movement of associated components such as movement of an associated robotic arm.

While steps can be taken to minimize the environmental disturbances, the minimization of some of these disturbances can also adversely affect the compounding process. For example, the air flow can produce a laminar flow to ensure the sterility of the source and final container consumables.

To minimize the inaccuracies of the gravimetric approach, steps are taken to stabilize the environment before taking a weight measurement. This includes minimizing movement and/or vibrations. This can be accomplished by maintaining other components of a compounding station motionless during the entire time a weight measurement is taken. In embodiments in which the compounding station includes a robotic arm, this robotic arm can be held motionless the entire time a weigh occurs. To further minimize disturbances during weighing, a compounding station can be closed to eliminate any disturbances from air movement. This can prevent loading consumables during a weighing process. Further, and as the static charge from a syringe can cause the scale to be inaccurate, before taking the weight of the empty scale, a syringe can be held sufficiently far away from the scale such that the static charge on the syringe doesn't interfere with the scale. These tight constraints on weighing a syringe (air flow, stable environment, closed chamber, and static charge interference prevention) significantly decrease compounding throughput, or in other words, the number of syringes that can be sterilely compounded in a given time period is reduced. The impact on throughput is especially severe as some implementations require multiple weights to determine the amount of liquid in a syringe, and specifically can require four weight measurements to determine the amount of liquid that has been aspired into the syringe, namely, two measurements to determine the empty syringe weight and two measurements to determine the loaded syringe weight.

Embodiments of the present address these shortcomings via the use of image analysis to determine a volume of aspirated liquid in a syringe. This includes aspirating liquid into a syringe, positioning the syringe in a predetermined location for imaging, capturing an image of the syringe, and evaluating the image of the syringe to determine the volume of aspirated liquid in the syringe. Embodiments as disclosed herein provide significant benefits, namely, improving throughput and improving accuracy of compounding.

In some aspects, embodiments of the present address these shortcoming via the use of computer vision. Computer vision is a field of artificial intelligence (AI) in which one or more machine learning models, which models can be deep learning models, neural networks, or the like, are utilized to extract, identify, recognize, and/or understand objects, features, and/or items in one or more images, videos, or the like.

More specifically, computer vision is the scientific field that focuses on how machines interpret visual information, such as images and videos. It can comprise one or more traditional image analysis algorithms and/or neural network based algorithms. Traditional algorithms are programmed while neural network based algorithms are trained. The training is called machine learning. During learning the neural networks are trained using a set of images with the targeted objects for object detection, classification, etc. In embodiments disclosed herein, one or more machine learning models, and specifically, one or more neural networks can be trained with images of, for example, a plunger seal, a meniscus, and/or the like so that the trained models can identify the plunger seal, meniscus, and/or the like.

In traditional machine vision, the features that are used to detect or identify an object of interest have to be selected by the vision engineer. In some embodiments utilizing machine learning, the algorithm automatically learns what to look for in each class by analyzing sample images where the targeted objects are masked. However, the training set is usually rather large and includes images as many as practically possible, including images of variations of the expected targets. If conditions of the environment change, for example if light, background, and/or the like change, the algorithm may need to be retrained. There many implementations of machine learning neural networks: TensorFlow, Yolo, U-Net, DeepLab, etc. Some of those are referred to as “deep learning” techniques. “Deep learning” is a type of machine learning that uses artificial neural networks to teach machines to learn and make decision.

In some embodiments, machine learning based on computer vision in which a machine learning model automatically learns and/or traditional computer vision in which a vision engineers selects features can be utilized to perform image segmentation described herein and/or to identify the plunger seal and/or the meniscus.

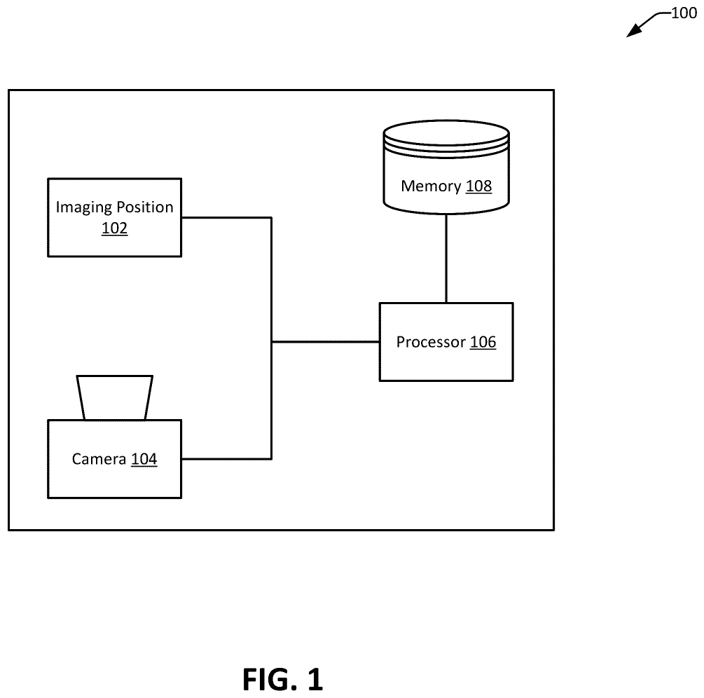

With reference now to FIG. 1, a schematic depiction of one embodiment of a system 100 for vision-based automated syringe volume measurement is shown. The system 100 can be a compounding station. The system 100 can include an imaging position 102. The imaging position 102 can be a predetermined position at which a syringe can be imaged. In some embodiments, the imaging position 102 can include a position and/or an orientation such as a vertical orientation. In some embodiments, the imaging position 102 can include a feature such as a fixture, a grabber, or the like which can secure the syringe in the imaging position 102 and specifically in the desired location and/or orientation. In some embodiments, the feature can include a robotic arm and/or robotic grabber which can grab a syringe and can hold the syringe in the desired location and/or orientation.

The system 100 can include a camera 104. The camera 104 can comprise a high-resolution camera that can be configured to capture an image of a syringe in the imaging position 102. In some embodiments, the camera 104 can comprise a 2 megapixel camera. In some embodiments, this configuring can include orienting the camera 104 such that the field of view of the camera 104 includes the imaging position 102.

In some embodiments, the camera 104 can be configured to provide 10 pixels of resolution for each millimeter of the syringe. In other words, the camera 104 can positioned with respect to the imaging position 102, can have lenses, and/or can have sufficient resolution such that each millimeter of the syringe is captured with at least 10 pixels in an image generated by the camera 104.

The system 100 can include one or more processors 106. The processor 106 can include, for example, logic circuitry and/or one or several integrated circuits such as, for example, a central processing unit (CPU), a graphics processing unit (GPU), or the like. In some embodiments, the processor 106 can be co-located with other components of the system 100, can be located remote from the other components of the system 100, and/or can be provided as a cloud-based service. The processor 106 can be configured to execute computer readable instructions which can be stored in the memory 108. In some embodiments, the process 106 can be communicatively coupled with one or both of the imaging position 102 and/or the camera 104. In some embodiments, the processor 106 can be configured to receive information from one or both of the imaging position 102 and/or the camera 104 and/or to provide one or several instructions controlling operation of one or both of the imaging position 102 and/or the camera 104. These instructions can include, for example, instructions the imaging position 102 to grab a syringe, to aspirate a volume of liquid into the syringe, to the position the syringe for imaging, to move a plunger of the syringe, and/or to dispense the syringe. In some embodiments, and as will be discussed at greater length below, the processor 106 can be configured to evaluate the captured image of the syringe to determine the liquid volume contained in the syringe.

The memory 108 can be any desired memory including, for example, main memory, auxiliary memory, or the like. The memory 108 can include volatile memory and/or non-volatile memory. In some embodiments, the memory 108 can be co-located with other components of the system 100, can be located remote from the other components of the system 100, and/or can be provided as a cloud-based service. The memory can store instructions executable by the processor 106 to cause the processor to take one or several actions.

In some embodiments, the memory 108 can comprise one or several databases. These databases can include, for example, one or several databases configured to contain information identifying one or several syringes and/or syringe types, identifying a model correlating distance to volume for one or several syringe types, images captured by the camera 104, volume determinations for one or several syringes, or the like.

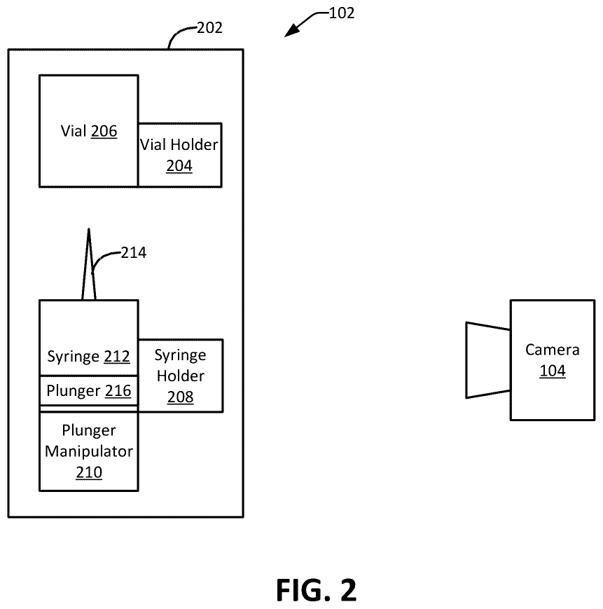

With reference now to FIG. 2, a schematic depiction of a camera 104 positioned to image a syringe in a syringe loading station 202 is shown. The syringe loading station 202 can be communicatively coupled with the processor 106 such that the syringe loading station 202 operates according to instructions received from the processor 106.

In some embodiments, the imaging position 102 can include the syringe loading station and, in some embodiments, the syringe loading station 202 can include the imaging position.

The syringe loading station 202 can hold and/or manipulate a syringe 212, can manipulate a plunger 216 of the syringe 212 to thereby aspirate liquid and/or gas into the syringe 212 and/or to dispense or expel liquid and/or gas from the syringe 212. In some embodiments, and as shown in FIG. 2, the syringe loading station 202 can include a vial holder 204. The vial holder can be configured to receive and/or hold a vial 206 containing liquid such as a medication for loading into a syringe 212.

The syringe loading station 202 can include a syringe holder 208. The syringe holder 208 can be configured to hold a syringe 212. In some embodiments, the syringe holder 208 can be configured to hold the syringe 212 at a desired location such as the imaging position 102 and/or to move the syringe 212 between one or several locations and/or stations within the compounding station 100. In some embodiments, the syringe holder 208 can comprise a computer-controlled grabber and/or arm, and specifically can comprise a robotic arm and/or robotic grabber. In some embodiments, the syringe holder 208 can be configured to grab a syringe, move the syringe to a location for aspirating liquid into the syringe 212, move the syringe 212 to the imaging position 102 for image capture, move the syringe 212 to a location for adjusting the liquid volume in the syringe 212, and move the syringe 212 to a dispensing location.

The syringe loading station 202 can further include a plunger manipulator 210. The plunger manipulator 210 can be configured to manipulate the plunger 216, and specifically to move the plunger 216 within the syringe 212 to thereby aspirate liquid and/or gas into the syringe 212 and/or to dispense liquid and/or gas from the syringe 212.

In some embodiments, the syringe 212 can include a needle 214. The needle 214 can define a lumen through which liquid and/or gas can be aspirated into the syringe 212 and/or dispensed from the syringe 212.

In some embodiments, the syringe holder 208 can position the syringe 212 with response to the vial 206 and the syringe 212 and/or the vial 206 can be moved with respect to each other such that the needle 214 is inserted into the vial 206. The plunger manipulator 210 can manipulate the plunger 216 to aspirate liquid from the vial 206 into the syringe 212. After a desired volume of liquid is aspirated into the syringe 212, the syringe and/or the vial 206 can be moved with respect to each other such that the needle 214 is either retracted from the vial 206 and/or positioned within a portion of the vial filled by air. The plunger manipulator 210 can manipulate the plunger 216 to aspirate air into the syringe 212 to thereby create a bubble, referred to herein as an air gap, separated the liquid in the syringe 212 from a top of the syringe 212.

After the air gap has been created, the syringe 212 can be positioned in the imaging position 102, and an image of the syringe can be captured by the camera. This image can be subsequently evaluated and/or analyzed by the processor 106 to thereby determine a liquid volume contained in the syringe 212. If this liquid volume deviates from a desired liquid volume, then the syringe 212 can be positioned to either aspirate further liquid from the vial 206 and/or to dispense liquid from the syringe 212. Liquid can then be aspirated into the syringe 212 from the vial 206 or can be dispensed from the syringe 212 to achieve the desired syringe liquid volume. After the aspirating and/or dispensing, a new image of the syringe can be captured, and the image can be evaluated to determine the volume of the liquid contained in the syringe 212. These steps can be repeated until the syringe 212 contains the desired volume, at which point the syringe can be dispensed from the compounding station.

In the event that the syringe 212 contains the desired liquid volume, then the size of the air gap can be determined, and the syringe can be positioned to expel the air forming the air gap. After the air forming the air gap has been dispensed from the syringe 212, the syringe 212 can be dispensed from the compounding station.

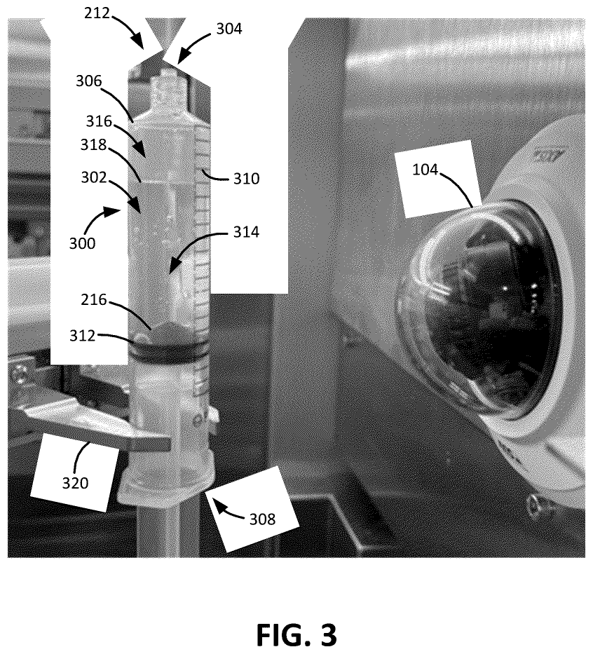

With reference now to FIG. 3, a depiction of a syringe 212 held by a robotic-arm and being imaged by the camera 104 is shown. The syringe 212 includes a syringe body 300 defining an internal volume 302. The syringe body 300 having a syringe tip 304, a syringe top 306, and a syringe bottom 308. The syringe tip 304 is the extreme point of the syringe 212 to which the needle 214 connects. The syringe top 306 is the top of the internal volume 302 of the syringe 212. The syringe body 300 can further include one or several graduation markings 310 which can indicate a volume of liquid contained in the syringe 212.

The syringe 212 further includes the plunger 216 which is moveable within the internal volume 302 of the syringe body 300 to aspirate liquid and/or gas into the internal volume 302 of the syringe 212 and/or to dispense and/or expel liquid and/or gas from the internal volume 302 of the syringe 212. A portion of the plunger 216 engages with the syringe body 300 to form a seal 312.

As seen in FIG. 3, when the internal volume 302 of the syringe 212 is partially filled with liquid, the internal volume 302 can include a fluid portion 314 and an air gap 316. The upper limit of the fluid portion 314 can be defined by a liquid meniscus 318.

As further seen in FIG. 3, the syringe 212 can be held by a robotic arm 320. The robotic arm 320 can be the syringe holder 208 and/or can be part of the syringe holder 208. The robotic arm 320 of FIG. 3 is holding the syringe 212 in front of the camera 104, and specifically is holding the syringe 212 at the imaging position 102 such that the syringe can be imaged by the camera 104.

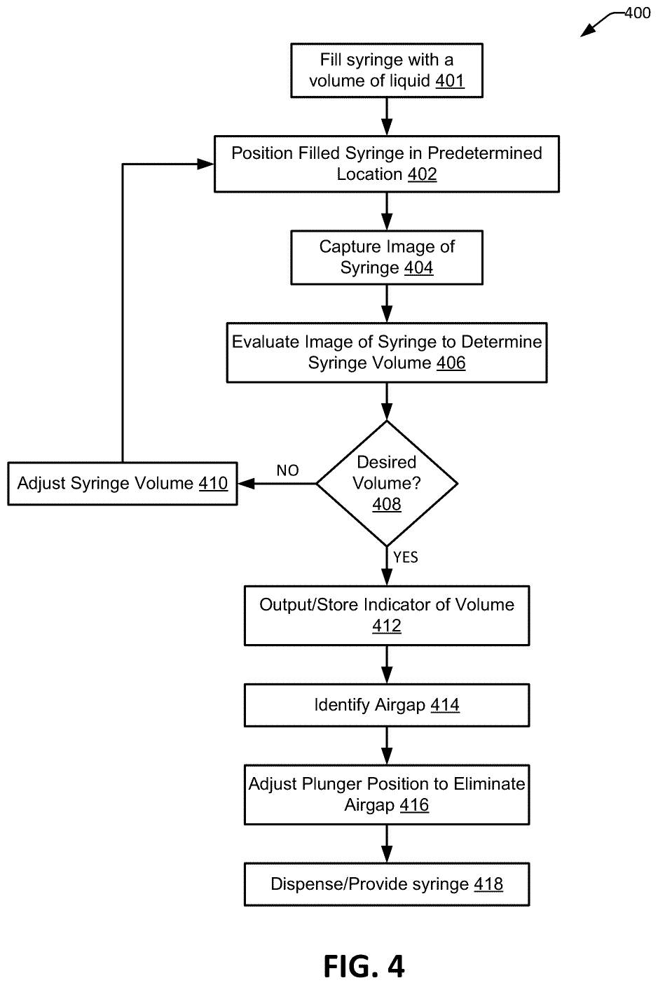

With reference now to FIG. 4, a flowchart illustrating one embodiment of a process 400 for vision-based automated syringe volume measurement is shown. The process 400 can be performed using some or all of the features and/or components of some or all of FIGS. 1 through 3. The process 400 begins at block 401, wherein the syringe 212 is filled with a volume of liquid. In some embodiments, the step of block 401 can include receiving an/or grabbing the syringe 212, and identifying the syringe 212. This can include, in some embodiments, the robotic arm 320 grabbing a syringe 212 and moving the syringe 212 to the syringe loading station 202. The syringe 212 can be identified to determine one or more attributes of the syringe 212 such as, for example, a relationship between a measure distance in the syringe 212 and a volume of the syringe 212. In some embodiments, identifying the syringe 212 can include identifying a maximum volume of the syringe 212.

When the syringe 212 is received at the loading station 202, syringe 212 can be manipulated such that the syringe 212 is fluidly coupled with the vial 206, which coupling can be achieved via the insertion of the needle 214 of the syringe 212 into the pile 206. The plunger manipulator 210 can retract the plunger 216 to thereby draw fluid from the vial 206 into the syringe 212.

In some embodiments, step 401 can further include creating an air gap in the syringe 212. This can include, for example, after filling the syringe 212 with the desired volume of liquid from the vial 206, repositioning the syringe 212 such that the needle 214 can aspirate air. This can include advancing the needle 214 further into the vial 206 such that the needle is located in an air pocket within the vial 206, or withdrawing the needle 214 from the vial 206. Once the needle 214 is in its desired location, the plunger manipulator 210 can retract the plunger 216 to draw air into the syringe 212 to thereby create the air gap.

At block 402, the filled syringe 212 is positioned in a predetermined location for imaging. In some embodiments, this predetermined location can be the imaging position 102. In some embodiments, the syringe 212 can be positioned at the predetermined location by the syringe holder 208, and specifically by the robotic arm 320.

At block 400 for an image of the syringe 212 is captured. In some embodiments, the image of the syringe 212 can be captured by the camera 104 controlled according to signals received from the processor 106.

At block 406, the image of the syringe 212 is evaluated to determine the syringe volume. In some embodiments, this evaluation can include locating one or several features of the syringe 212 such as, for example, locating one or more of the tip 304 of the syringe 212, the top 306 of the syringe 212, the plunger 216 of the syringe 212, the seal 312, the liquid meniscus 318, or the like. In some embodiments, distances between one or several of these features can be determined, which distance can then be used to determine a volume of liquid in the syringe 212.

A decision step 408, it is determined if the volume in the syringe is the desired volume. In some embodiments, this can include comparing the volume of liquid determined to be in the syringe 212 in step 406 to a desired volume for the syringe 212. In some embodiments determining if the volume in the syringe 212 is a desired volume can include determining a difference between the volume of liquid determined to be in the syringe 212 and a desired volume of liquid for the syringe 212 can be determined, and this difference can be compared to one or several threshold values. In some embodiments, if this difference is greater than one or several of the threshold values, then it can be determined that the actual volume of liquid in the syringe is not the desired volume, and the process 400 can advance to step 410 wherein the syringe volume is adjusted.

In some embodiments, adjusting the syringe volume can include determining, with the processor 106, how to change the volume of liquid in the syringe. This can include aspirating more liquid into the syringe or dispensing an/or dispelling liquid from the syringe. In some embodiments, the processor can direct the plunger manipulator 210 to manipulate the plunger 216 to thereby adjust the volume of liquid in the syringe 212 to match the desired volume. After the volume of liquid in the syringe 212 has been adjusted, the process 400 can return to block 402 and proceed as outlined above.

Returning again to decision step 408, if it is determined that the volume of liquid in the syringe is the desired volume, then the process 400 proceeds to block 412 wherein an indicator of the volume in the syringe is outputted and/or stored. In some embodiments, this can include outputting information relating to the volume of the syringe to the user and/or storing the indicator of the volume in the memory 108.

At step 414 the air gap in the syringe is identified. In some embodiments, this can include evaluating the image of the syringe to identify the air gap and to determine the volume of air. This evaluation can utilize feature locations identified in step 406. In some embodiments, for example, step 414 can be performed as a part of step 406, or can be performed separate from step 406. Based on the location of features of the syringe 212, the volume of the air gap can be identified.

At step 416, the air gap is illuminated. In some embodiments, the air gap can be eliminated by adjusting the plunger 216 to dispense an/or expel the air from the syringe 212. In some embodiments, this can include controlling the plunger manipulator 210 to adjust the plunger 216 such that the air gap is illuminated.

At step 418, the syringe 212 is dispensed an/or provided. In some embodiments, this can include dispensing the syringe 212 to the user, and/or providing the syringe 212 for further use by the compounding station 100 such as, for example, for compounding an IV bag.

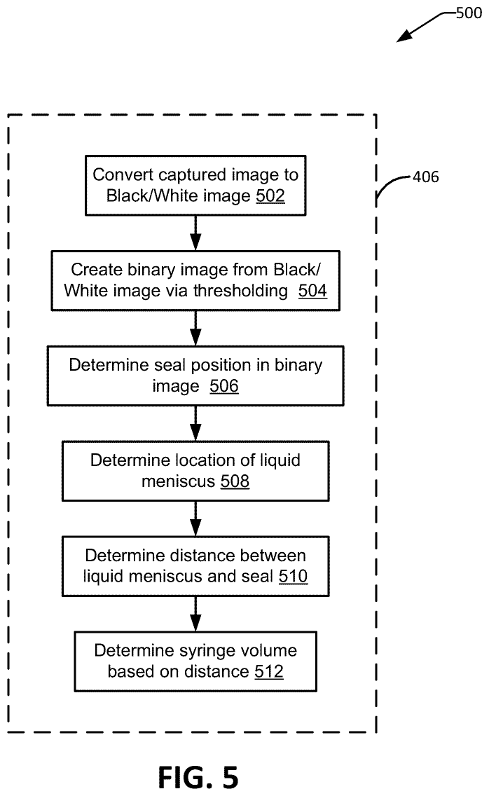

With reference now to FIG. 5, a flowchart illustrating one embodiment of a process 500 for evaluating an image of a syringe to determine a syringe volume is shown. The process 500 can be performed as a part of, or in the place of block 406 of FIG. 4.

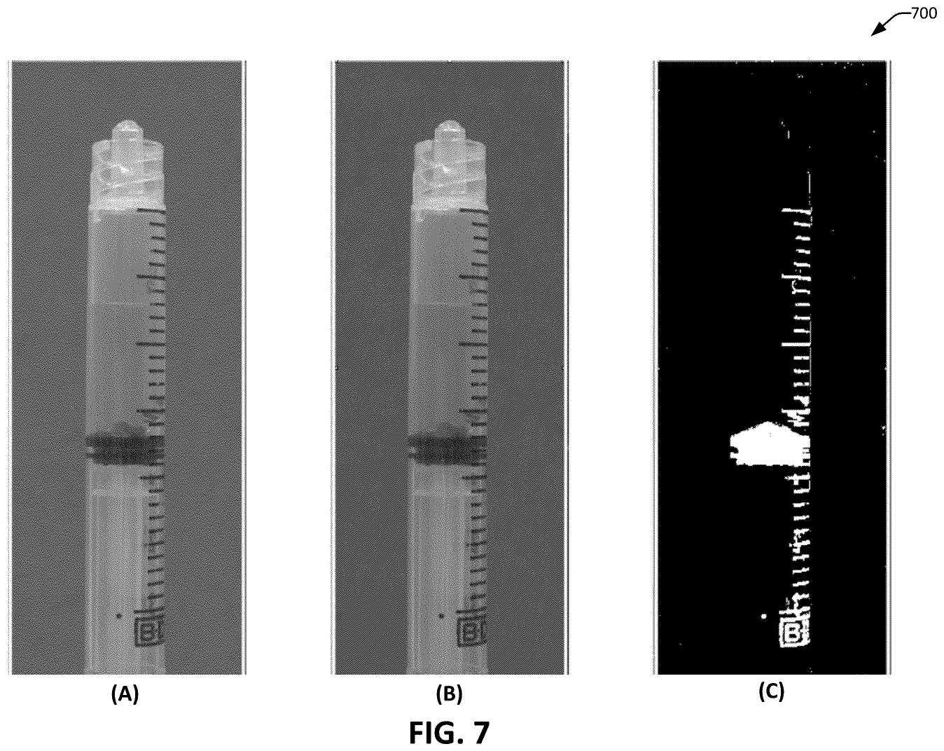

The process 500 begins at block 502 where the captured image of the syringe is converted to a black-and-white (“B/W”) image. In some embodiments, for example, the captured image can be an RGB image. The result of block 502 is depicted in FIG. 7, which includes images showing an image of a syringe in raw form, as a black-and-white image, and as a binary image. Specifically, as seen in FIG. 7, image (A) is the RGB image, and image (B) is the converted B/W image.

At step 500 for, a binary image can be created from the B/W image generated in step 502. In some embodiments, the binary image can be created via thresholding. In some embodiments, this thresholding can be performed according to an Isodata algorithm, an Otsu algorithm, or the like. The result of block 504 is depicted in FIG. 7, which includes images showing an image of a syringe in raw form, as a black-and-white image, and as a binary image. Specifically, as seen in FIG. 7, image (C) is the binary image.

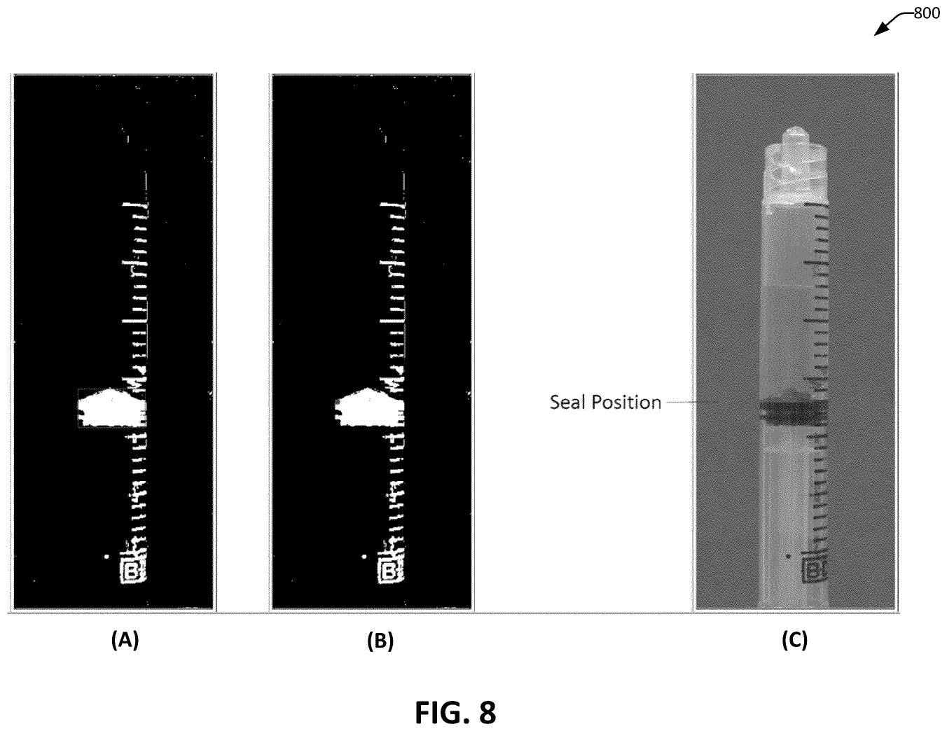

At step 506 the seal position is determined from the binary image. In some embodiments, the seal position can be determined utilizing computer vision including, for example, traditional computer vision and/or machine learning-based computer vision. In some embodiments, this can include performing blob analysis on the binary image to identify the plunger 216. Once the plunger 216 is identified, the top ring edge of the plunger 216 can be identified, the location of which top ring edge of the plunger 216 can be the seal location. The determining of the seal positioned is depicted in FIG. 8, which includes images depicting one embodiment of a process for identifying a seal position. As seen in FIG. 8, the plunger is identified in the binary image as shown in (A). Once the plunger 216 has been identified, the top ring edge is identified, as indicated by the red dot in (B). The location of this top ring edge is identified as the seal position as shown in (C).

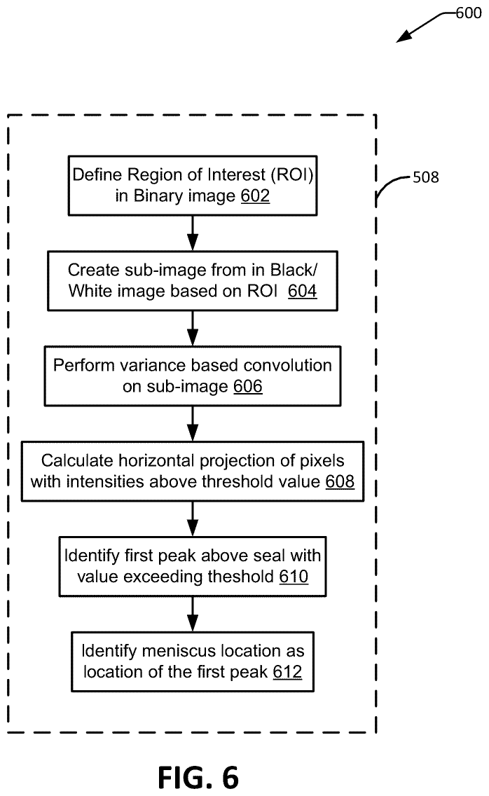

At step 508, the location of the liquid meniscus 318 is determined. In some embodiments, the location of the liquid meniscus can be determined utilizing computer vision including, for example, traditional computer vision and/or machine learning-based computer vision. The determination of the location the liquid meniscus 318 will be discussed in greater length below with respect to FIG. 6.

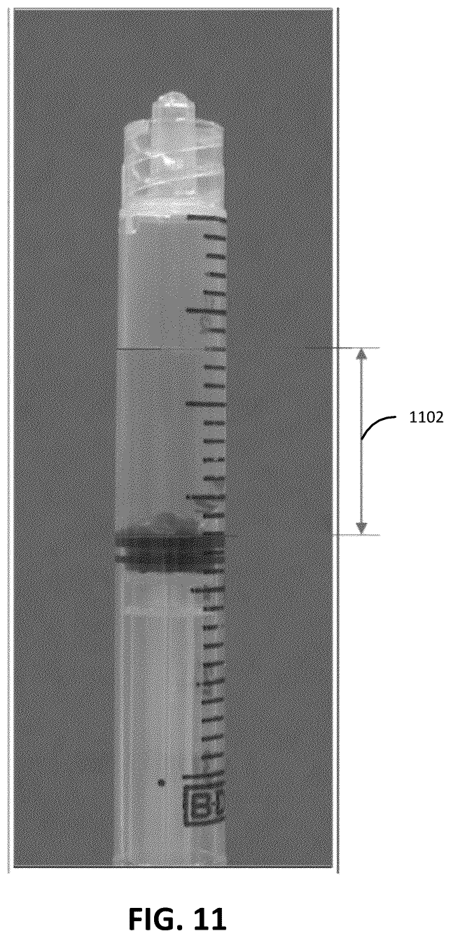

At step 510, a distance between the liquid meniscus 318 and the seal 312 is determined. In some embodiments, this determination can include identifying a shortest line between the liquid meniscus 318 and the seal 312 and determining an/or counting the pixels in this line. In some embodiments, and as discussed above, the camera 104 can be configured and/or have sufficient resolution such that each millimeter of distance at the syringe 212 corresponds to at least 10 pixels. This distance of 1102 is indicated in the image depicting the distance between the seal position and the liquid meniscus location of a syringe of FIG. 11.

At step 512, a volume of the syringe is determined based on the determined distance of step 510. In some embodiments, this volume can be determined based on a model and/or correlation for the syringe. For example, in a syringe 212 of a given maximum capacity, each pixel in the straight line between the liquid meniscus 318 and the seal 312 can correspond to a known volume. The eyes, the volume of the syringe 212 can be determined by multiplying the number of pixels in the straight line between the liquid meniscus 318 and the seal 312 with the known volume per pixel.

With reference now to FIG. 6, a flowchart illustrating one embodiment of a process 600 for determining a location of a liquid meniscus in the syringe 212 is shown. The process 600 can be performed as a part of, or in the place of the step of block 508 of FIG. 5.

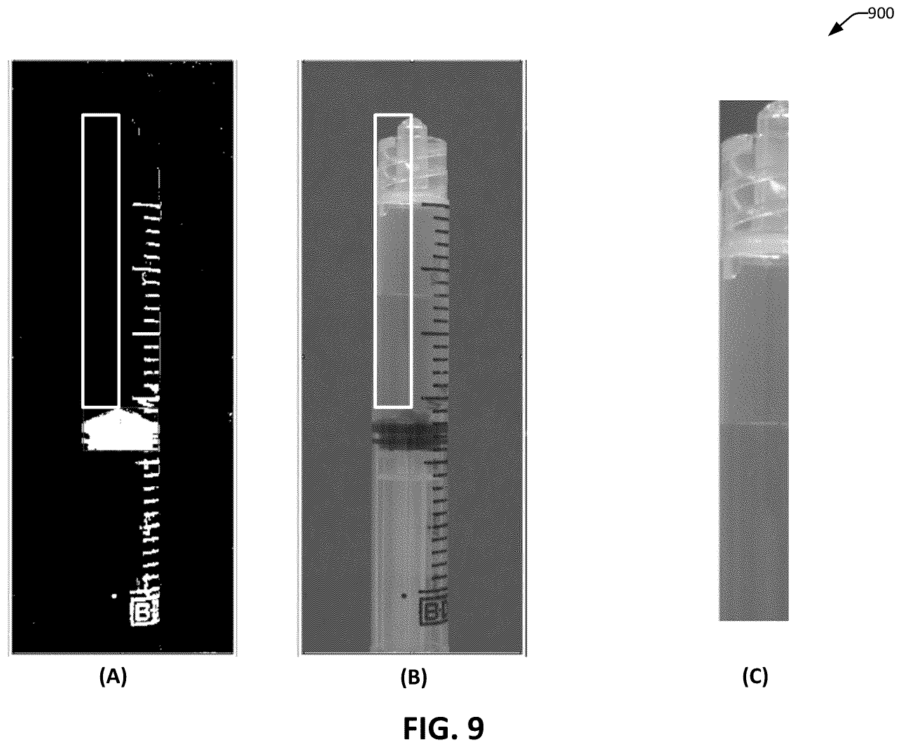

At block 602 a region of interest (“ROI”) is defined in the binary image or alternatively in the B/W image. In some embodiments, the ROI can be defined based on the location of the seal 312 and the graduation markings 310. Specifically, the ROI can be defined as a vertical portion of the image extending from the seal 312 to the tip 304 and/or top 306 of the plunger 212 and not including the graduation markings 310. The ROI is the area included within the white box in (A) and (B) of FIG. 9, which figure includes images depicting one embodiment of a process of creating a sub-image.

At block 604, and based on the defined ROI, a sub-image corresponding to the ROI is created from the B/W image. This sub-image includes, in some embodiments, all or portions of the image found in the ROI. The sub-image is indicated in (C) of FIG. 9.

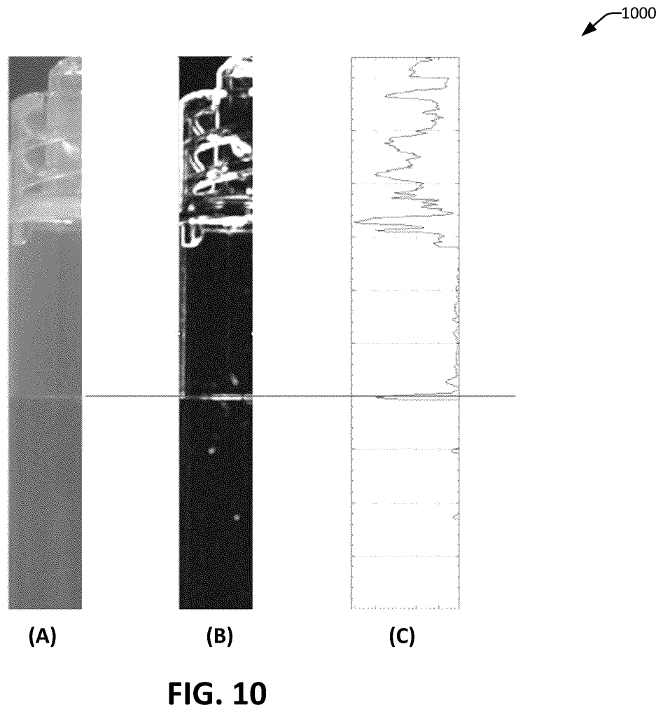

At block 606, a convolution is performed on the sub image. In some embodiments, this convolution can be a variance-based convolution. This convolution can include, for example, simple blurring (smoothing) of the image where each pixel intensity of the output image is equal to the average of intensities of pixels within a predefined vicinity region. For example, the pixel intensity of the output image can be equal to the average intensities of pixels within a 9-pixel neighborhood in the case of 3×3 convolution kernel, or equal to the average intensities of pixels within a 25-pixel neighborhood in the case of 5×5 convolution kernel size. In some embodiments, this convolution can include, for example, simple blurring (smoothing) of the image where each pixel intensity of the output image is equal to the variance of intensities of pixels within a predefined vicinity region. For example, the pixel intensity of the output image can be equal to the variance of intensities of pixels within a 9-pixel neighborhood in the case of 3×3 convolution kernel, or equal to the variance of intensities of pixels within a 25-pixel neighborhood in the case of 5×5 kernel size. The result of the variance-based convolution is shown in (B) of FIG. 10, which figure includes images depicting one embodiment of a process for determining a liquid meniscus location.

At block 608, a horizontal projection of pixels with intensities above a threshold value is calculated. In some embodiments, the horizontal projection can be calculated based on the result of the convolution, and specifically based on the result of the variance-based convolution. The horizontal projection of pixels with intensities above a threshold value is shown in (C) of FIG. 10.

At block 610, a first peak above the seal 312 and with a value exceeding a threshold value is identified. In some embodiments, this threshold value is defined as a portion of the maximum projection height, or in other words, as a portion of the maximum horizontal projection of pixels. In some embodiments, this threshold value is defined as one half of the maximum projection height.

At block 612 the location of the liquid meniscus 318 is identified as the location of the first peak identified in block 610. The location of the liquid meniscus 318 can, in some embodiments, be stored in memory 108.

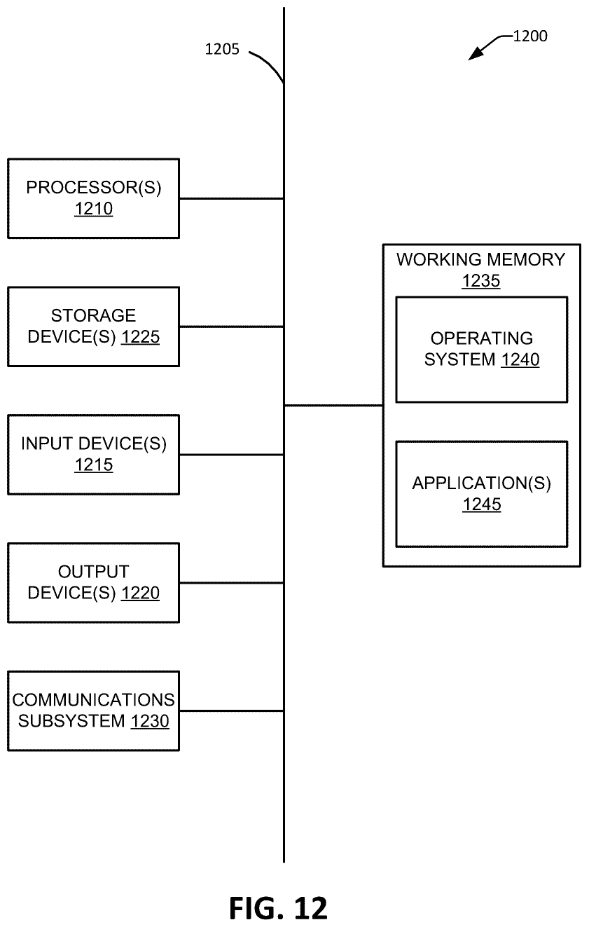

With reference now to FIG. 12, a computer system may be incorporated as part of the previously described computerized devices. For example, computer system 1200 can represent some of the components of system 100, processor 106, and/or other computing devices described herein. FIG. 12 provides a schematic illustration of one embodiment of a computer system 1200 that can perform the methods provided by various other embodiments, as described herein. FIG. 12 is meant only to provide a generalized illustration of various components, any or all of which may be utilized as appropriate. FIG. 12, therefore, broadly illustrates how individual system elements may be implemented in a relatively separated or relatively more integrated manner.

The computer system 1200 is shown comprising hardware elements that can be electrically coupled via a bus 1205 (or may otherwise be in communication, as appropriate). The hardware elements may include a processing unit 1210, including without limitation one or more processors, such as one or more special-purpose processors (such as digital signal processing chips, graphics acceleration processors, and/or the like); one or more input devices 1215, which can include without limitation a keyboard, a touchscreen, receiver, a motion sensor, an imaging device, and/or the like; and one or more output devices 1220, which can include without limitation a display device, a speaker, and/or the like.

The computer system 1200 may further include (and/or be in communication with) one or more non-transitory storage devices 1225, which can comprise, without limitation, local and/or network accessible storage, and/or can include, without limitation, a disk drive, a drive array, an optical storage device, a solid-state storage device such as a random access memory (“RAM”) and/or a read-only memory (“ROM”), which can be programmable, flash-updateable and/or the like. Such storage devices may be configured to implement any appropriate data stores, including without limitation, various file systems, database structures, and/or the like.

The computer system 1200 might also include a communication interface 1230, which can include without limitation a modem, a network card (wireless or wired), an infrared communication device, a wireless communication device and/or chipset (such as a Bluetooth™ device, an 502.11 device, a Wi-Fi device, a WiMAX device, an NFC device, cellular communication facilities, etc.), and/or similar communication interfaces. The communication interface 1230 may permit data to be exchanged with a network (such as the network described below, to name one example), other computer systems, and/or any other devices described herein. In many embodiments, the computer system 1200 will further comprise a non-transitory working memory 1235, which can include a RAM or ROM device, as described above.

The computer system 1200 also can comprise software elements, shown as being currently located within the working memory 1235, including an operating system 1240, device drivers, executable libraries, and/or other code, such as one or more application programs 1245, which may comprise computer programs provided by various embodiments, and/or may be designed to implement methods, and/or configure systems, provided by other embodiments, as described herein. Merely by way of example, one or more procedures described with respect to the method(s) discussed above might be implemented as code and/or instructions executable by a computer (and/or a processor within a computer); in an aspect, then, such special/specific purpose code and/or instructions can be used to configure and/or adapt a computing device to a special purpose computer that is configured to perform one or more operations in accordance with the described methods.

A set of these instructions and/or code might be stored on a computer-readable storage medium, such as the storage device(s) 1225 described above. In some cases, the storage medium might be incorporated within a computer system, such as computer system 1200. In other embodiments, the storage medium might be separate from a computer system (e.g., a removable medium, such as a compact disc), and/or provided in an installation package, such that the storage medium can be used to program, configure and/or adapt a special purpose computer with the instructions/code stored thereon. These instructions might take the form of executable code, which is executable by the computer system 1200 and/or might take the form of source and/or installable code, which, upon compilation and/or installation on the computer system 1200 (e.g., using any of a variety of available compilers, installation programs, compression/decompression utilities, etc.) then takes the form of executable code.

Substantial variations may be made in accordance with specific requirements. For example, customized hardware might also be used, and/or particular elements might be implemented in hardware, software (including portable software, such as applets, etc.), or both. Moreover, hardware and/or software components that provide certain functionality can comprise a dedicated system (having specialized components) or may be part of a more generic system. For example, a risk management engine configured to provide some or all of the features described herein relating to the risk profiling and/or distribution can comprise hardware and/or software that is specialized (e.g., an application-specific integrated circuit (ASIC), a software method, etc.) or generic (e.g., processing unit 1210, applications 1245, etc.) Further, connection to other computing devices such as network input/output devices may be employed.

Some embodiments may employ a computer system (such as the computer system 1200) to perform methods in accordance with the disclosure. For example, some or all of the procedures of the described methods may be performed by the computer system 1200 in response to processing unit 1210 executing one or more sequences of one or more instructions (which might be incorporated into the operating system 1240 and/or other code, such as an application program 1245) contained in the working memory 1235. Such instructions may be read into the working memory 1235 from another computer-readable medium, such as one or more of the storage device(s) 1225. Merely by way of example, execution of the sequences of instructions contained in the working memory 1235 might cause the processing unit 1210 to perform one or more procedures of the methods described herein.

The terms “machine-readable medium” and “computer-readable medium,” as used herein, refer to any medium that participates in providing data that causes a machine to operate in a specific fashion. In an embodiment implemented using the computer system 1200, various computer-readable media might be involved in providing instructions/code to processing unit 1210 for execution and/or might be used to store and/or carry such instructions/code (e.g., as signals). In many implementations, a computer-readable medium is a physical and/or tangible storage medium. Such a medium may take many forms, including but not limited to, non-volatile media, volatile media, and transmission media. Non-volatile media include, for example, optical and/or magnetic disks, such as the storage device(s) 1225. Volatile media include, without limitation, dynamic memory, such as the working memory 1235. Transmission media include, without limitation, coaxial cables, copper wire, and fiber optics, including the wires that comprise the bus 1205, as well as the various components of the communication interface 1230 (and/or the media by which the communication interface 1230 provides communication with other devices). Hence, transmission media can also take the form of waves (including without limitation radio, acoustic and/or light waves, such as those generated during radio-wave and infrared data communications).

Common forms of physical and/or tangible computer-readable media include, for example, a magnetic medium, optical medium, or any other physical medium with patterns of holes, a RAM, a PROM, EPROM, a FLASH-EPROM, any other memory chip or cartridge, a carrier wave as described hereinafter, or any other medium from which a computer can read instructions and/or code.

The communication interface 1230 (and/or components thereof) generally will receive the signals, and the bus 1205 then might carry the signals (and/or the data, instructions, etc. carried by the signals) to the working memory 1235, from which the processor(s) 1205 retrieves and executes the instructions. The instructions received by the working memory 1235 may optionally be stored on a non-transitory storage device 1225 either before or after execution by the processing unit 1210.

The methods, systems, and devices discussed above are examples. Some embodiments were described as processes depicted as flow diagrams or block diagrams. Although each may describe the operations as a sequential process, many of the operations can be performed in parallel or concurrently. In addition, the order of the operations may be rearranged. A process may have additional steps not included in the figure. Furthermore, embodiments of the methods may be implemented by hardware, software, firmware, middleware, microcode, hardware description languages, or any combination thereof. When implemented in software, firmware, middleware, or microcode, the program code or code segments to perform the associated tasks may be stored in a computer-readable medium such as a storage medium. Processors may perform the associated tasks.

It should be noted that the systems and devices discussed above are intended merely to be examples. It must be stressed that various embodiments may omit, substitute, or add various procedures or components as appropriate. Also, features described with respect to certain embodiments may be combined in various other embodiments. Different aspects and elements of the embodiments may be combined in a similar manner. Also, it should be emphasized that technology evolves and, thus, many of the elements are examples and should not be interpreted to limit the scope of the invention.

Specific details are given in the description to provide a thorough understanding of the embodiments. However, it will be understood by one of ordinary skill in the art that the embodiments may be practiced without these specific details. For example, well-known structures and techniques have been shown without unnecessary detail in order to avoid obscuring the embodiments. This description provides example embodiments only, and is not intended to limit the scope, applicability, or configuration of the invention. Rather, the preceding description of the embodiments will provide those skilled in the art with an enabling description for implementing embodiments of the invention. Various changes may be made in the function and arrangement of elements without departing from the spirit and scope of the invention.

The methods, systems, devices, graphs, and tables discussed above are examples. Various configurations may omit, substitute, or add various procedures or components as appropriate. For instance, in alternative configurations, the methods may be performed in an order different from that described, and/or various stages may be added, omitted, and/or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims. Additionally, the techniques discussed herein may provide differing results with different types of context awareness classifiers.

While illustrative and presently preferred embodiments of the disclosed systems, methods, and machine-readable media have been described in detail herein, it is to be understood that the inventive concepts may be otherwise variously embodied and employed, and that the appended claims are intended to be construed to include such variations, except as limited by the prior art.

Unless defined otherwise, all technical and scientific terms used herein have the same meaning as commonly or conventionally understood. As used herein, the articles “a” and “an” refer to one or to more than one (i.e., to at least one) of the grammatical object of the article. By way of example, “an element” means one element or more than one element. “About” and/or “approximately” as used herein when referring to a measurable value such as an amount, a temporal duration, and the like, encompasses variations of +20% or +10%, +5%, or +0.1% from the specified value, as such variations are appropriate to in the context of the systems, devices, circuits, methods, and other implementations described herein. “Substantially” as used herein when referring to a measurable value such as an amount, a temporal duration, a physical attribute (such as frequency), and the like, also encompasses variations of +20% or +10%, +5%, or +0.1% from the specified value, as such variations are appropriate to in the context of the systems, devices, circuits, methods, and other implementations described herein.

As used herein, including in the claims, “and” as used in a list of items prefaced by “at least one of or “one or more of indicates that any combination of the listed items may be used. For example, a list of “at least one of A, B, and C” includes any of the combinations A or B or C or AB or AC or BC and/or ABC (i.e., A and B and C). Furthermore, to the extent more than one occurrence or use of the items A, B, or C is possible, multiple uses of A, B, and/or C may form part of the contemplated combinations. For example, a list of “at least one of A, B, and C” may also include AA, AAB, AAA, BB, etc.

Having described several embodiments, it will be recognized by those of skill in the art that various modifications, alternative constructions, and equivalents may be used without departing from the spirit of the invention. For example, the above elements may merely be a component of a larger system, wherein other rules may take precedence over or otherwise modify the application of the invention. Also, a number of steps may be undertaken before, during, or after the above elements are considered. Accordingly, the above description should not be taken as limiting the scope of the invention.

Also, the words “comprise”, “comprising”, “contains”, “containing”, “include”, “including”, and “includes”, when used in this specification and in the following claims, are intended to specify the presence of stated features, integers, components, or steps, but they do not preclude the presence or addition of one or more other features, integers, components, steps, acts, or groups.

What is claimed is:

1. A method of syringe volume measurement comprising:
filling a volume of liquid into a syringe, wherein the volume of liquid is unknown;
positioning the syringe for imaging with a high-resolution camera;
capturing an image of the syringe with the high-resolution camera; and
evaluating the image of the syringe to determine the volume of liquid in the syringe.

2. The method of claim 1, further comprising filling air into the syringe to create an air gap between a meniscus of the liquid in the syringe and a top of the syringe.

3. The method of claim 2, wherein positioning the syringe for imaging comprises positioning the syringe in a vertical orientation with the top above a plunger of the syringe.

4. The method of claim 3, wherein positioning the syringe for imaging comprises holding the syringe with a robotic arm.

5. The method of claim 2, wherein evaluating the image comprises:
determining a seal position; and
determining the volume of liquid in the syringe based on the seal position.

6. The method of claim 5, wherein evaluating the image further comprises:
determining a location of a liquid meniscus in the image of the syringe; and
determining a distance between the location of the liquid meniscus and the seal 3 position, wherein the volume of liquid in the syringe is determined based on the distance between the location of the liquid meniscus and the seal position.

7. The method of claim 6, wherein determining the distance between the location of the liquid meniscus and the seal position comprises counting a number of pixels in a shortest straight line between the seal position and the location of the liquid meniscus.

8. The method of claim 6, wherein determining the location of the liquid meniscus comprises:
defining a region of interest (“ROI”) in the image of the syringe; and
creating a sub-image based on the image of the syringe and on the ROI.

9. The method of claim 8, wherein determining the location of liquid meniscus comprises:
performing a variance-based convolution on the sub-image;
calculating horizontal projection of pixels with intensities above a threshold value; and
identifying a first peak in the horizontal projection of pixels above the seal position.

10. The method of claim 9, further comprising identifying the location of the first peak in the horizontal projection of pixels as the location of the liquid meniscus.

11. The method of claim 6, wherein evaluating the image further comprises:
converting the image of the syringe to a black-and-white image; and
creating a binary image from the black-and-white image.

12. The method of claim 11, wherein the binary image is created from the black-and-white image via thresholding.

13. Th method of claim 11, wherein the seal position is determined in the binary image.

14. The method of claim 6, further comprising:
identifying the air gap based on the image of the syringe; and
adjusting the plunger position in the syringe to eliminate the air gap.

15. The method of claim 14, wherein identifying the air gap comprises:
evaluating the image of the syringe to identify a top of the syringe; and
determining a distance between the liquid meniscus and the top of the syringe.

16. A system for syringe volume measurement comprising:
a syringe loading station;
a syringe holder;
a high-resolution camera configured to image the syringe loading station; and
a processor configured to:
fill with the syringe loading station a volume of liquid into a syringe, wherein the volume of liquid is unknown;
position with the syringe holder the syringe for imaging with the camera;
capture with the camera an image of the syringe with the high-resolution camera; and
evaluate the image of the syringe to determine the volume of liquid in the syringe.

17. The system of claim 16, wherein the processor is further configured to fill air into the syringe with the syringe loading station to create an air gap between a meniscus of the liquid in the syringe and a top of the syringe.

18. The system of claim 17, wherein positioning the syringe for imaging comprises positioning the syringe in a vertical orientation with the top above a plunger of the syringe.

19. The system of claim 18, wherein positioning the syringe for imaging comprises holding the syringe with a robotic arm.

20. The system of claim 17, wherein evaluating the image comprises:
determining a seal position; and
determining the volume of liquid in the syringe based on the seal position.

21. The system of claim 20, wherein evaluating the image further comprises:
determining a location of a liquid meniscus in the image of the syringe; and
determining a distance between the location of the liquid meniscus and the seal position, wherein the volume of liquid in the syringe is determined based on the distance between the location of the liquid meniscus and the seal position.

22. The system of claim 21, wherein determining the distance between the location of the liquid meniscus and the seal position comprises counting a number of pixels in a shortest straight line between the seal position and the location of the liquid meniscus.

23. The system of claim 21, wherein determining the location of the liquid meniscus comprises:
defining a region of interest (“ROI”) in the image of the syringe; and
creating a sub-image based on the image of the syringe and on the ROI.

24. The system of claim 23, wherein determining the location of liquid meniscus comprises:
performing a variance-based convolution on the sub-image;
calculating horizontal projection of pixels with intensities above a threshold value; and
identifying a first peak in the horizontal projection of pixels above the seal position.

25. The system of claim 24, wherein the processor is further configured to identify the location of the first peak in the horizontal projection of pixels as the location of the liquid meniscus.

26. The system of claim 21, wherein evaluating the image further comprises:
converting the image of the syringe to a black-and-white image; and
creating a binary image from the black-and-white image.

27. The system of claim 26, wherein the binary image is created from the black-and-white image via thresholding.

28. The system of claim 26, wherein the seal position is determined in the binary image.

29. The system of claim 21, wherein the processor is further configured to:
identify the air gap based on the image of the syringe; and
adjust the plunger position in the syringe to eliminate the air gap.

30. The system of claim 29, wherein identifying the air gap comprises:
evaluating the image of the syringe to identify a top of the syringe; and
determining a distance between the liquid meniscus and the top of the syringe.
